(19) United States
(12) Patent Application Publication
(10) Pub. No.: US 2017/0368135 A1
(43) Pub. Date: Dec. 28, 2017

(54) HERBAL DRUG COMPOSITION CONTAINING PROBIOTIC AND OPTIONALLY PREBIOTIC AND/OR ACTIVE PHARMACEUTICAL INGREDIENT

(71) Applicant: VITALIVA, LLC, New Brunswick, NJ (US)

(72) Inventors: Neil Shivraj DAVEY, Gaithersburg, MD (US); Neil RUSTGI, New Brunswick, NJ (US); Priya PURI, Waxhaw, NC (US); Sonya Raj DAVEY, Gaithersburg, MD (US); Vinod RUSTGI, New Brunswick, NJ (US)

(73) Assignee: VITALIVA, LLC

(21) Appl. No.: 15/351,674

(22) Filed: Nov. 15, 2016

Related U.S. Application Data
(60) Provisional application No. 62/355,089, filed on Jun. 27, 2016.

(51) Int. Cl. A61K 38/06; A61K 36/9066; A61K 36/79; A61K 36/484; A61K 36/28; A61K 31/14; A61K 36/258; A61K 36/233; A61K 35/747; A61K 35/745; A61K 36/9068; A61K 36/481

(52) U.S. Cl. CPC A61K 38/063; A61K 36/9068; A61K 36/9066; A61K 36/79; A61K 36/484; A61K 36/481; A61K 31/14; A61K 36/258; A61K 36/233; A61K 35/747; A61K 35/745; A61K 36/28

(57) ABSTRACT

The embodiments herein relate to the combination of herbal and probiotic (optionally with prebiotic) treatment for chronic liver diseases to be novel and more effective. Embodiments herein relate to a composition comprising a probiotic and a medicinal herb (and optionally a prebiotic), wherein the composition is effective in treating a liver disease such as inflammation of liver in a novel approach to disease management. The combination of a prebiotic, a probiotic and a medicinal herb constitutes a symbiotic formulation. Clinical and animal data exists to show efficacy of these combinations. The ingredients, as well as the categories, in the combination are novel. Using a combination of twelve herbs and three probiotics, a highly efficacious and novel supplement is disclosed herein. Embodiments of the compositions herein could further include homeopathic drugs and an active pharmaceutical ingredient such as an allopathic drug.

### RELATED APPLICATIONS

This application claims benefit to U.S. Provisional Patent Application No. 62/355,089, filed Jun. 27, 2016, entitled, “HERBAL DRUG COMPOSITION CONTAINING PROBIOTIC AND OPTIONALLY PREBIOTIC AND/OR ACTIVE PHARMACEUTICAL INGREDIENT,” which is incorporated herein by reference in its entirety.

### FIELD OF INVENTION

The invention is related to herbal drug compositions containing a probiotic, and optionally a prebiotic and/or an active pharmaceutical ingredient, wherein the prebiotic and the probiotic combine to preferably form a symbiotic formulation.

### BACKGROUND

Chronic liver diseases (CLD) affect lions of Americans, and hundreds of millions of people worldwide. A long list of conditions can lead to chronic liver diseases, including viral hepatitis, alcoholic hepatitis, non-alcoholic steatohepatitis (NASH), drag-induced liver injury, malnutrition or inherited metabolic disorders (e.g. hemachromatosis).

The American Liver Foundation describes the progression of liver diseases (http://www.liverfoundation.org/abouttheliver/info/progression/) as follows:

“There are many different types of liver disease. But no matter what type you have, damage to your liver is likely to progress in a similar way. Whether your liver is infected with a virus, injured by chemicals, or under attack from your own immune system, the basic danger is the same—that your liver will become so damaged that it can no longer work to keep you alive. Anything that keeps your liver from doing its job may put your life in danger.

### The Healthy Liver

Your liver helps fight infections and cleans your blood. It also helps digest food and stores energy for when you need it. A healthy liver has the amazing ability to grow back, or regenerate, when it is damaged. Anything that keeps your liver from doing its job—or from growing back after injury may put your life in danger.

### Inflammation

In the early stage of any liver disease, your liver may become inflamed. It may become tender and enlarged. Inflammation shows that your body is trying to fight an infection or heal an injury. But if the inflammation continues over time, it can start to hurt your liver permanently. When most other parts of your body become inflamed, you can feel it—the area becomes hot and painful. But an inflamed liver may cause you no discomfort at all.

If your liver disease is diagnosed and treated successfully at this stage, the inflammation may go away.

### Fibrosis

If left untreated, the inflamed liver will start to scar. As excess scar tissue grows, it replaces healthy liver tissue. This process is called fibrosis. (Scar tissue is a kind of fibrous tissue.). Scar tissue cannot do the work that healthy liver tissue can. Moreover, scar tissue can keep blood from flowing through your liver. As more scar tissue builds up, your liver may not work as well as it once did. Or, the healthy part of your liver has to work harder to make up for the scarred part. If your liver disease is diagnosed and treated successfully at this stage, there's still a chance that your liver can heal itself over time.

### Cirrhosis

But if left untreated, your liver may become so seriously scarred that it can no longer heal itself. This stage—when the damage cannot be reversed—is called cirrhosis.”

With the development of CLD, liver cirrhosis may ensue which can be complicated by liver cancer (hepatocellular carcinoma) or portal hypertension (in turn complicated by ascites, encephalopathy, gastrointestinal bleeding, hepatorenal syndrome). Liver transplantation is successful in a small subset of these patients impeded by the lack of donor livers.

Even though around 30% of adults in the United States have a form of liver disease, effective therapies are lacking for many liver diseases, whether acute or chronic, especially NASH. The most effective treatment for liver disease remains cutting back on alcohol consumption, improving the diet, and engaging in exercise regularly. Anti-viral therapies do exist for hepatitis B as well as hepatitis C, but even there, opportunities exist. Other than fundamental lifestyle changes and direct acting anti-viral therapies, there is no specific treatment that exists for many liver diseases today. The American Journal of Gastroenterology has reported that for liver diseases, 74% of patients reported using complementary and alternative medicines (CAM) in addition to the medications prescribed by their physician. It is thus desirable to create novel supplements that have high levels of efficacy in treating patients with various forms of liver disease. Herbal medicines offer inroads to improving the health of patients with acute and chronic liver disease. These medicines act through their anti-oxidant properties, reducing reactive oxygen species (ROS), improving response of liver cells to injury/infection through anti-apoptotic properties and other unrecognized mechanisms. Thus, there is a compelling need to apply herbal medicines in a rational and targeted fashion in these circumstances. More importantly, as inflammation of liver is an important step in the progression of liver disease, it is particularly pertinent to find a therapy that is targeted at preventing or treating inflammation of liver.

### SUMMARY OF INVENTION

Embodiments herein relate to a composition comprising a prebiotic, a probiotic and a medicinal herb, wherein the composition is effective in treating a liver disease or disorder in a human or an animal, and wherein the composition is a symbiotic formulation.

Preferably, the composition comprises the medicinal herb comprises fennel, fenugreek, or mixtures thereof.

Preferably, the composition further comprises a vitamin, a mineral supplement, a bulking agent, a hydrocolloid, a lipotrophic factor or mixtures thereof.

Preferably, the probiotic comprises Lactobacillus acidophilus, Bifidobacterium bindus, Lactobacillus rhamnosus or mixtures thereof.

Preferably, the prebiotic comprises a selectively fermented ingredient that allows specific changes, both in the composition and/or activity in a gastrointestinal microflora that confers benefits upon host well-being and health.

Preferably, the probiotic comprises a non-pathogenic microbial culture.

Preferably, the probiotic comprises a multiple mixed strain culture.

Preferably, the probiotic comprises genera Lactococcus, Lactobacillus, Pediococcus, Streptococcus, Propionibacterium, Brevibacterium, Penicillium, Saccharomyces, or mixtures thereof.

Preferably, the composition is of the type useful in treating a disease or disorder selected from the group consisting of anemia, arthritis, constipation, depression, diabetes, dyspepsia, hemorrhoids, hepatitis, hypertension, impotency, obesity, overweight, periodontal disease, and combinations thereof.

Preferably, the composition further comprises an active pharmaceutical ingredient.

Another embodiment relates to a composition comprising a prebiotic, a probiotic and a medicinal herb, wherein the composition is effective in treating a disease or disorder in a human or an animal, the disease being selected from the group consisting of tension, memory loss, joint stiffness, joint pain, swelling, liver disease, rectal bleeding, rectal pain, male impotency, loss of sex drive, high blood cholesterol, bleeding gums, bad breath, tooth ache, digestive disorder, bowel disorder, and combinations thereof, and wherein the composition is a symbiotic formulation.

Preferably, the composition further comprises an active pharmaceutical ingredient.

Another embodiment relates to a method of treating a liver disease or disorder in a human or an animal subject by administering to a subject in need of such treatment a composition in a known effective amount for treating the disease or disorder, wherein the method comprises administering to the subject need of treatment the composition of claim 1.

Preferably, the probiotic is selected from the group consisting of non-pathogenic members of the genera Lactococcus, Lactobacillus, Pediococcus, Streptococcus, Propionibacterium, Brevibacterium, Penicillium, and Saccharomyces, and mixtures thereof.

Preferably, the method further comprises, prior to the step of administering, culturing the probiotic; and drying the probiotic culture.

Preferably, the step of drying the probiotic culture is by a method selected from the group consisting of spray drying, heat drying, and freeze drying.

Preferably, the method further comprises, prior to the step of administering, blending the composition and the probiotic; and forming the mixture into combined dosage units.

Preferably the step of administering is performed by simultaneously administering separate dosage units.

Preferably, the probiotic is administered in a quantity from 0.01% to 90% by weight of the composition.

Preferably, the composition further comprises an active pharmaceutical ingredient.

### BRIEF DESCRIPTION OF DRAWINGS

The patent or application file contains at least one drawing executed in color. Copies of this patent or patent application publication with color drawing(s) will be provided by the Office upon request and payment of the necessary fee.

FIG. 1 shows digital images of histopathology of control Liver 1C wherein the liver contains multifocal lobular areas of hepatocellular necrosis and loss, degeneration, and neutrophilic inflammatory infiltrate with mild portal neutrophilic inflammatory infiltrate and moderate portal lymphocytic inflammatory infiltrate.

FIG. 2 shows digital images of histopathology of control Liver 6C wherein inflammation, biliary hyperplasia, and fibrosis are not observed.

FIG. 3 shows digital images of histopathology of control Liver 10C wherein the images histologically similar to that of Liver 1C but inflammatory infiltrates and biliary hyperplasia are lessened.

FIG. 4 shows digital images of histopathology of one supplemental treated Liver 2 wherein one portal triad shows mild fibrosis.

FIG. 5 shows digital images of histopathology of one supplement treated Liver 3 wherein hepatocellular steatosis is diffuse within the liver of the supplement treated animals; inflammation, degeneration, and biliary hyperplasia are not identified in the supplement treated liver.

FIG. 6 shows digital images of histopathology of one supplement treated Liver 9 wherein hepatocellular steatosis is diffuse within the liver of the supplement treated animals; inflammation, degeneration, and biliary hyperplasia are not identified in the supplement treated liver.

### DETAILED DESCRIPTION

A variety of herbal treatments for liver disease have shown to be effective, as can he seen in patent applications CN103007195 A, WO2002032444 A1, US20130273182 A1, EP1328284 A1 (US counterparts: U.S. Pat. No. 6,455,078 B1 and U.S. Pat. No. 6,696,094 B2), WO2013048355 A2, CN101766785 A, CN103751605 A, WO2011080721 A2, and CA2425900 A1 (US counterparts: U.S. Pat. No. 8,431,620 B2 and US 20130209527 A1). Most of these patent applications involve Chinese herbal compositions for the treatment of liver disease.

Embodiments herein relate to compositions of one or more herbs as well as one or more prebiotics and one or more probiotics and therefore, preferably a symbiotic formulation, for the treatment of diseases, preferably liver diseases comprising inflammation of liver.

The term “inflammation” refers to the complex biological response of body tissues to harmful stimuli, such as pathogens, damaged cells, or irritants, and is a protective response involving immune cells, blood vessels, and molecular mediators. The function of inflammation is to eliminate the initial cause of cell injury, clear out necrotic cells and tissues damaged from the original insult and the inflammatory process, and to initiate tissue repair.

Inflammation can be classified as either acute or chronic. Acute inflammation is the initial response of the body to harmful stimuli and is achieved by the increased movement of plasma and leukocytes (especially granulocytes) from the blood into the injured tissues. A series of biochemical events propagates and matures the inflammatory response, involving the local vascular system, the immune system, and various cells within the injured tissue. Prolonged inflammation, known as chronic inflammation, leads to a progressive shift in the type of cells present at the site of inflammation, such as mononuclear cells, and is characterized by simultaneous destruction and healing of the tissue from the inflammatory process.

Inflammation is not a synonym for infection. Infection describes the interaction between the action of microbial invasion and the reaction of the body's inflammatory defensive response—the two components are considered together when discussing an infection, and the word is used to imply a microbial invasive cause for the observed inflammatory reaction. Inflammation on the other hand describes purely the body's immunovascular response, whatever the cause may be. But because of how often the two are correlated, words ending in the suffix -itis (which refers to inflammation) are sometimes informally described as referring to infection. For example, the word urethritis strictly means only “urethral inflammation”, but clinical health care providers usually discuss urethritis as a urethral infection because urethral microbial invasion is the most common cause of urethritis.

It is useful to differentiate inflammation and infection as there are many pathological situations where inflammation is not driven by microbial invasion—for example, atherosclerosis, type III hypersensitivity, trauma, ischaemia.

The term “inflammation of liver” refers to a disease of the liver characterized by the presence of inflammatory cells in the tissue of the organ. This disease is often called “hepatitis,” but not limited to just hepatitis. Inflammation of liver may occur without symptoms, but can lead to jaundice (a yellow discoloration of the skin, mucous membranes, and conjunctiva of the eyes), poor appetite, and fatigue. Depending on the cause, hepatitis can manifest either as an acute or as a chronic disease. Acute hepatitis can be self-limiting (resolving on its own), can progress to chronic hepatitis, or can cause acute liver failure in rare instances. Chronic hepatitis may have no symptoms, or may progress over time to fibrosis (scarring of the liver) and cirrhosis (chronic liver failure). Cirrhosis of the liver increases the risk of developing hepatocellular carcinoma (a form of liver cancer).

Worldwide, viral hepatitis is the most common cause, followed closely by alcoholic liver disease and non-alcoholic liver disease (NAFLD). Other less common causes of hepatitis include autoimmune diseases, ingestion of toxic substances, certain medications (such as acetaminophen), some industrial organic solvents, and plants.

1. Viral infection: Many viruses can cause liver inflammation and they are infectious and transmittable. Now we know, the major viral causes of hepatitis are HAV (hepatitis A virus), HBV (hepatitis B virus), HCV (hepatitis C virus), HDV (hepatitis delta virus), and HEV (hepatitis enteric or epidemic virus). HFV and HGV have also been described. Among them, HAV and HDV are usually self-limited infections. Chronic HBV and HCV infections on the other hand are the most common. It is estimated that 400 million worldwide have HBV and 170 million have HCV. These types of the hepatitis can progress to serious liver diseases, such as cirrhosis and liver cancer. Patients who have Hepatitis C should definitely have the HAV and HBV vaccinations in order to avoid co-infection with these two kinds of viral infections. Co-infections can dramatically accelerate the progression rate of the chronic condition.

2. Alcoholic hepatitis: Alcohol abuse can cause liver inflammation because alcohol and its metabolite are toxic to liver cells. Alcoholic liver diseases are three times more common than HCV infection. Thus, it is very important for patients with HCV to avoid alcohol.

3. Drug or chemical intoxication: Many drugs and chemicals can cause liver damage and induce hepatitis, such as carbon-tetrachloride, amethopterin, tetracycline, acetaminophen, fenoprofen, etc. The degree and severity of the liver damage is dependent dosage, the length of the course, and individual's genetic background. Long-term, certain drugs or chemicals can induce chronic hepatitis, even cirrhosis or accelerate progression to cirrhosis. Thus, people with hepatitis C should avoid taking unnecessary drugs and stop using any recreational chemicals. When there is a infection or condition that calls for antibiotics or pain-killers, the patient must also be careful when choosing the drugs and the amount of the drugs.

4. Others: Many other systemic infections can also infect the liver, such as EBV (Epstein-Barr virus) or bacterial infections, especially in immunocompromised individuals. They can also cause liver enzyme elevation and other liver dysfunctions. In these diseases, the liver may only be part of a systemic manifestation.

By medical definition, if the liver inflammation last less than six months, it is considered acute hepatitis; if the disease course lasts longer than six months, the liver inflammation is defined as chronic.

The compositions and methods of prevention or treatment of liver, including liver inflammation, according to embodiments herein are applicable for all of the above forms and causes of hepatitis.

Diagnosis of liver inflammation (e.g., hepatitis) is made on the basis of some or all of the following: a patient's signs and symptoms, medical history including sexual and substance use history, blood tests, imaging, and liver biopsy. In general, for viral hepatitis and other acute causes of hepatitis, the patient's blood tests and clinical picture are sufficient for diagnosis.

Common imaging modalities for the diagnosis of hepatitis include ultrasound, CT, and MRI. All of these modalities can identify steatosis (fatty changes) of the liver tissue and nodularity of the liver surface suggestive of cirrhosis. CT and especially MRI are able to provide a higher level of detail, allowing visualization and characterize such structures as vessels and tumors within the liver. Liver biopsy is considered the most definitive diagnostic test to assess inflammation and fibrosis of the liver. Liver biopsy is the gold standard for establishing the diagnosis of inflammation of liver, as histopathologic analysis (histopathology) is able to reveal the precise extent and pattern of inflammation and fibrosis.

Histopathology refers to the microscopic examination of tissue in order to study the manifestations of disease. Specifically, in clinical medicine, histopathology refers to the examination of a biopsy or surgical specimen by a pathologist, after the specimen has been processed and histological sections have been placed onto glass slides.

Staining of processed histology slides can be done to slides processed by the chemical fixation or frozen section slides. To see the tissue under a microscope, the sections are stained with one or more pigments. The aim of staining is to reveal cellular components; counterstains are used to provide contrast.

The most commonly used stain in histopathology is a combination of hematoxylin and eosin (often abbreviated H&E). Hematoxylin is used to stain nuclei blue, while eosin stains cytoplasm and the extracellular connective tissue matrix pink. Other compounds used to color tissue sections include safranin, Oil Red O, congo red, silver salts and artificial dyes. Masson's trichrome is used to emphasize fibrosis tissue or scarring as that area of a slide is seen under the microscope as blue in contrast to tissue such as hepatocytes (functioning liver cells) which are seen as pink by H&E.

Typically, a pathologist examines the histological slides under a microscope. This medical diagnosis is formulated as a pathology report describing the histological findings and the opinion of the pathologist.

Oil Red O staining was also used in the tests undertaken herein in addition to H&E and Masson's trichrome. Oil Red O is one of the dyes that have to be performed on fresh samples as alcohol fixation removes most lipids. It can make fat more visible in pathology sections

Though the use of probiotics in the treatment of liver disease is not as well characterized as that of herbs, the use of such microorganisms can be seen in patent applications US20050180962 A1, CN10284558 A, and EP1409010 A1 (US counterpart: US 20040047868 A1). U.S. Pat. No. 6,080,401, entitled “Herbal and Pharmaceutical Drugs Enhanced with Probiotics” discloses an improved preparation of a drug having a combination of a probiotic and a drug (herbal, allopathic or periodontal) other than antibiotics, antibodies, and drugs inhibitory to the viability of the probiotic.

The embodiments herein relate to the combination of herbal and probiotic, and optionally prebiotic, for the treatment for chronic liver diseases that is both novel and more effective than the current standard of care. Embodiments herein relate to a composition comprising a prebiotic, a probiotic and medicinal herbs, wherein the composition is effective in treating a liver disease. The combination of a prebiotic, a probiotic (preferably, a symbiotic formulation) and medicinal herbs is a novel approach to disease management. According to embodiments herein, clinical and animal data have been generated to show efficacy of these combinations. The ingredients, as well as the categories, in the combination are novel.

For example, in a formulation according to embodiments herein, using a combination of sixteen herbs and four probiotics as well as a prebiotic, a highly efficacious and novel supplement is disclosed herein. Embodiments of the compositions herein could further include homeopathic drugs and an active pharmaceutical ingredient such as an allopathic drug.

Studies according to embodiments herein demonstrate that the synergistic effect of multiple herbs and probiotics demonstrate extremely strong potentials for treatment. The combined use of herbs and probiotics and a prebiotic in this manner is completely novel:

Herbs refer to plants used for medicine, i.e., medicinal herbs. Herbs could include leaves, roots, flowers, seeds, resin, root bark, inner bark (and cambium), berries and sometimes the pericarp or other portions of the plant. In the embodiments herein, herbs generally are plants that contain phytochemicals that have effects on the body. Herbal ingredients include: Caper bush, Chicory, Black nightshade, Tamarisk, Schisandra Chinensis 1.5 grams, Bupleurum Chinense, Ginger, Ginseng, Licorice, Turmeric, Silyrnarin 260 mg, Glutathione, Neem, Astragalus root, Choline, and Crataegus Pinnatifida.

TABLE 1
List of Herbs in Formulation with Benefits for Treating Liver Disease.

| Herbs | Benefits |
|---|---|
| 1. Caper bush | C. spinosa, commonly known as caper bush, has been proven to show |
| (10-80 g/kg body | protective measures against hepatotoxicity. In an animal model study of |
| weight, preferably, 30-40 g/kg | hepatotoxicity induced by CCl₄, it was shown that mice that were |
| body weight) | induced with ethanolic root bark extract of C. spinosa in addition to |
| | CCl₄ had lower levels of alanine transaminase (ALT) and aspartate |
| | transaminase (AST) compared to those without the C. spinosa. This |
| | signifies the protection of hepatic cells, as high levels of ALT and AST |
| | are common indicators of liver disease. Thus, the induction of caper |
| | bush proves to be hepatoprotective in mice with hepatotoxicity. |
| 2. Chicory | The effect of chicory seed extract on diabetic rats with hepatic |
| (3-5 g/day, preferably | steatosis was studied. Various dosages (1.25, 2.5 and 5 mg/ml) of |
| 4 g/day) | chicory seed extract were induced in rats, simultaneously and non- |
| | simultaneously. The expression levels of sterol regulatory element- |
| | binding protein-1c (SREBP-1c) and peroxisome proliferator-activated |
| | receptor alpha (PPARα) was then determined. SREBP-1c plays an |
| | important role between lipids and insulin regulation, and PPARα is an |
| | important factor in SREBP-1c regulation. Hepatic steatosis decreases |
| | SREBP-1c and PPARα expression. It was found that in simultaneous |
| | treatment, chicory seed extract prevented the down-regulation of |
| | SREBP-1c and PPARα in HepG2 cells and diabetic liver cells. In |
| | non-simultaneous treatment, chicory seed extract restored SREBP-1c |
| | and PPARα to normal levels. |
| 3. Black nightshade | S. nigrum, commonly known as black nightshade, possesses reparative |
| (0.2 g/kg body weight- | qualities in rats with liver damage due to CCl₄ induction. Specifically, |
| 1.5 g/kg body | CCl₄-induced rats were treated with S. nigrum at a dose of 0.2 g/kg |
| weight, preferably | body weight for six weeks. This restored the increased levels of hepatic |
| 1 g/kg body weight) | enzymes (glutamic-oxaloacetic acid transaminase, glutamic-pyruvic |
| | acid transaminase, and alkaline phosphatase). A higher dose of 0.5 and |
| | 1.0 g/kg body weight resulted in the restoration of superoxide dismutase |
| | and glutathione peroxidase levels, indicating the repair of hepatic tissue |
| | damage due to CCl₄ induction. Treatment with S. nigrum also reduced |
| | ALT levels and normalized bilirubin levels. This evidence suggests that |
| | S. nigrum can protect against liver damage in rats. |
| 4. Tamarisk | Tamarisk extract contains several antioxidants, suggesting medical |
| (3-10 g/day, | benefits. Evidence suggests both restorative and preventative properties |
| preferably 7 g/day) | of tamarisk in relation to liver disease. Studies have shown treatment |
| | with tamarisk to normalize serum protein and cholesterol levels, which |
| | are affected by viral hepatitis. Further, it has been shown that tamarisk |
| | inhibits cytotoxicity and hepatic oxidative stress by restoring hepatic |
| | antioxidant enzymes, and stopping chemically-induced liver tumor |
| | proliferation. Data also suggests a chemopreventive effect of tamarisk, |
| | including the ability to restore cellular antioxidants and prevent |
| | mutagen update or DNA incorporation. |
| 5. Schisandra | “Schisandra Chinensis (SC), a traditional herbal medicine, has been |
| Chinensis | prescribed for patients suffering from various liver diseases, including |
| (1-2 g/kg body weight, | hepatic cancer, hypercholesterolemia, and CCl₄-induced liver injury.” A |
| preferably 1.5 g/kg | further animal model study found that SC also has a protective effect on |
| body weight) | alcohol-induced fatty liver. Rats induced with ethanol experienced |
| | increased liver total cholesterol (TC) and triglyceride (TG) levels. This |
| | effect was reversed in the rats induced with SC extract in addition to |
| | ethanol. Furthermore, it was found that the addition of SC extract also |
| | significantly increased phospho-AMPK and PPARα expression in |
| | hepatic tissue of alcoholic rats, suggesting that SC has the ability to |
| | prevent alcohol-induced fatty liver. |
| 6. Bupleurum | Data suggests that Bupleurum Chinense contains both preventative and |
| Chinense | therapeutic qualities for liver disease. CCl₄-induced liver disease was |
| (100-500 mg/kg | administered. The effects of CCl₄ include oxidative stress, which can |
| body weight, | lead to lipid peroxidation and affect the levels of antioxidant enzymes |
| preferably 400 mg/kg | in the liver. Administration of Bupleurum Chinense three days before |
| body weight) | a single dose of CCl₄ significantly lowered AST and ALT levels, |
| | important hepatic enzyme markers indicating liver disease. The |
| | pretreatment with Bupleurum Chinense also suppresses lipid |
| | peroxidation and reverses the antioxidant enzymes to normal levels. |
| | Further, treatment with Bupleurum Chinense showed to heal or partially |
| | heal liver lesions, such as ballooning degeneration, necrosis, hepatitis, |
| | and portal triaditis. |
| 7. Ginger | There is evidence supporting the ability of ginger to prevent or inhibit |
| (250 mg/day-4 g/day, | the progression of non-alcoholic fatty liver disease (NAFLD). Data |
| preferably 2 g/day) | shows that ginger can activate the peroxisome proliferator-activated |
| | receptor γ which induces adiponectin and down-regulates pro- |
| | inflammatory cytokines. This changes the balance between adiponectin |
| | and tumor necrosis factor-α and promotes the effects of antioxidants |
| | and antidyslipidemic properties, which reduces hepatic triglyceride |
| | content. This reduction can prevent hepatic steatosis. |
| 8. Ginseng | Treatment with preparations containing ginseng extract can improve |
| (1-2 g/day, preferably | detoxifying activity of the liver in elderly patients with toxin-induced |
| 1.5 g/day) | chronic liver disease. Data shows that ginseng extract modify |
| | bromsulphthalein retention and blood zinc levels and reduce serum bile |
| | acids and gamma-glutamyl transpeptidase before and after a fatty meal. |
| | Another study revealed that two months of oral ginseng improves lipid |
| | profiles in NAFLD of rats. Ginseng increases Natural Killer Cell |
| | activity in NAFLD of rats. Two months of oral ginseng inhibits NASH |
| | in NAFLD of rats. |
| 9. Licorice | Licorice reduces alanine aminotransferase (ALT) and aspartate |
| (1-3 g/day, preferably, | aminotransferase (AST) levels in the blood. High levels of ALT and |
| 2 g/day) | AST are both strongly correlate with liver damage, injury, or disease. |
| | Data shows that 2 g oral administration of licorice root extract per day |
| | for two months decreased ALT from 64.09 to 51.27□IU/mL and |
| | AST from 58.18 to 49.45□IU/mL, which were statistically significant |
| | (p□<□0.001 and p□<□0.001). |
| 10. Turmeric | Turmeric prevents alcoholic liver disease by suppressing induction of |
| (50-100 mg/kg of | NF-κB-dependent genes. Data on curcimin, an antioxidant found in the |
| body weight/day, | dietary spice turmeric, shows that curcimin administration prevented |
| preferably 75 mg/kg | phsyciological changes induced by alcohol. Curcumin blocks |
| of body weight/day) | endotoxin-mediated activation of NF-κB and suppresses the expression |
| | of cytokines, chemokines, COX-2, and iNOS in Kupffer cells. |
| | Inhibition of endotoxin and Kupffer cells in turn decreases alcohol liver |
| | disease by suppressing NF-κB-dependent genes. |
| 11. Silymarin | Silymarin is the most commonly used herb for treating liver disease. |
| (120-300 mg/day, | Silymarin reduces alanine aminotransferase (ALT) and aspartate |
| preferably 260 mg/day) | aminotransferase (AST) levels in the blood. High levels of ALT and |
| | AST are both strongly correlate with liver damage, injury, or disease. |
| | Data demonstrates that mean ALT and AST levels deceased from 103.1 |
| | to 41.4 and 53.7 to 29.1 IU/mL, respectively which was statistically |
| | significant (P < 0.001 & P < 0.001). In the control group, the decrease in |
| | mean ALT and AST, with decrease of 7.8 and 2.2 IU/mL, respectively, |
| | was not statistically significant. (Mohammadi, 2008) Additionally, in |
| | patients treated with Silymarin favorable secondary endpoints include: |
| | lowered plasma levels of ALT, 4-hydroxynonenal (a marker of lipid |
| | peroxidation), procollagen. type I and mRNA of α(I) procollagen (2 |
| | markers of fibrogenesis) and hepatic total triglycerides. Thus, there is |
| | evidence that Silymarin is a hepatoprotective that can retard alcohol |
| | liver disease progression. |
| 12. Glutathione | Data demonstrates that experimental models of alcoholic liver disease |
| (75 mg-150 mg/day, | and associated with mitochondrial lipid peroxidation and progression of |
| preferably 100 mg/day) | liver damage are associated with a progressive and selective depletion |
| | of mitochondrial glutathione. Thus, it can be concluded that by |
| | increasing levels of glutathione using a supplement can retard the |
| | progress of alcohol liver disease. |
| 13. Neem | Neem extract protects patients from hepatic cell damage caused by |
| (400-600 mg/kg body | paracetamol induction. Neem extract decreases glutamate oxaloacetate |
| weight, preferably 500 mg/kg | transaminase, glutamate pyruvate transaminase, acid phosphatase and |
| body weight) | alkaline phosphatase (all of which are elevated by paracetamol). The |
| | antihepatotoxic effects are likely due to an alteration in |
| | biotransformation of the toxic substances resulting in decreased |
| | formation of reactive metabolites. |
| 14. Astragalus root | Astragalus root decreased liver fibrosis in rats induced with carbon |
| (150-200 mg/kg of | tetrachloride. Astragalus root reduced liver damage and the symptoms |
| body weight, | of liver fibrosis. Astragalus root decreases elevation of serum |
| preferably 160 mg/kg | transaminase activities, hyaluronic acid, laminin and procollagen type |
| of body weight) | III levels, and contents of hydroxyproline in liver tissue by |
| | approximately 30-60%. Also, Astragalus root decreased the elevation |
| | of TGF-β1 by 47.7% and 53.1%, respectively. |
| 15. Choline | Data demonstrates that decreased choline intake significantly increases |
| (0.5 g-3.5 g/day, | liver disease in both patients and mouse models. A choline deficient |
| preferably 2 g/day) | diet in mice resulted in liver disease. Additionally decrease choline |
| | intake is significantly associated with increased fibrosis in |
| | postmenopausal women with NAFLD. Thus, this data demonstrates that |
| | increasing choline intake can prevent liver disease progression, making |
| | choline a key inuedient in the supplement. |
| 16. Crataegus | Crataegus Pinnatifida extract reversed increased plasma total |
| Pinnatifida | cholesterol and high density lipoprotein cholesterol induced by high |
| (50-150 mg/kg of | cholesterol diets in mice. Additionally, it demonstrated hepatoprotective |
| body weight/day, | function by reducing lipid content in the liver. Crataegus Pinnatifida |
| preferably 100 mg/kg | extract may provide its cholesterol lowering effect by up-regulating |
| of body weight/day) | hepatic CYP7A1 mRNA expression, which enhances bile acid |
| | biosynthesis. Thus, Crataegus Pinnatifida extract can improve liver |
| | disease by relieving hypercholesterolaemia-related complications. |

In conjunctions with herbs, both prebiotics and probiotics can be added to the supplements of the embodiments herein.

Probiotics are live micro-organisms which, when administered in adequate amounts, confer a health benefit on a host. The host can be humans and/or animals. Probiotics have to be alive when administered. Only products containing live organisms shown in reproducible human studies to confer a health benefit can actually claim to be a probiotic.

The probiotic candidate must be a taxonomically defined microbe or combination of microbes (genus, species and strain level). The effects of probiotic could be strain-specific, requiting identification of the strain, i.e. genotypic and phenotypic characterization of the tested microorganism. Probiotics must be safe for their intended use.

Probiotics have to be supplied in adequate amounts which may be defined as the amount able to trigger the targeted effect on the host. It depends on strain specificity, process and matrix as well as the targeted effect. In the preferred embodiments herein, benefits are demonstrated when probiotics were ingested in a concentration of around 10⁷ to 10⁸ microorganisms per gram of the probiotic, with a serving size of around 100 to 200 mg per day of the probiotic.

Probiotics could include: Lactobacillus; Bacteroidetes; Proteobacteria;and Enterobacteriaceae. Additional probiotics could include: Bacillus coagulans GBI-30, 6086; Bifidobacterium longum subsp. in antis 35624; Lactobacillus acidophilus NCFM; Lactobacillus paracasei St11 (or NCC2461); Lactobacillus johnsonii La1(=Lactobacillus LC1, Lactobacillus johnsonii NCC533); Lactobacillus plantarum 299v; Lactobacillus reuteri ATCC 55730 (Lactobacillus reuteri SD2112); Lactobacillus reuteri Protectis (DSM 17938, daughter strain of ATCC 55730); Lactobacillus reuteri Prodentis (DSM 17938/ATCC 55730 and ATCC PTA 5289 in combination) for oral health; Saccharomyces boulardii Lactobacillus rhamnosus GR-1® & Lactobacillus reuteri RC-14®; Lactobacillus acidophilus CL1285 & Lactobacillus casei LBC80R; and Lactobacillus plantarum HEAL 9 & Lactobacillus paracasei 8700:2. Some additional forms of lactic acid bacteria include: Lactobacillus bulgaricus; Streptococcus thermophilus; and “Lactobacillus byidus”—a new genus Bifidobacterium.

Fermentation and greater energy extraction of carbohydrates by bacteria can lead to liver injury. Some probiotics that can be added to prevent liver disease are Lactobacillus, Bacteroidetes, Proteobacteria, and Enterobacteriaceae.

Strains of lactobacilli reduce endotoxemia and alcohol-induced liver injury. Lactobacilli exhibited a positive effect on liver lipid peroxidation through an anti-oxidative stress activity by enhancing the liver antioxidant defense system. In addition, it inhibited insulin resistance, decreased fatty acid synthase, and increased expression of fasting-induced adipose factors in the liver. Lactobacilli have been demonstrated to affect the level of lipopolysaccharide in NAFLD mice by lowering intestinal permeability. Thus, Lactobacilli can be effective in preventing NAFLD induced by a high fat diet. In embodiments herein, the formulation includes the two different Lactobacilli (acidophilus and rhamnosus).

According to embodiments herein, the probiotic Bifidobacterium plays an important role in energy balance and to help reduce TNF-alpha, CRP, AST, HOMA-IR, serum endotoxin and steatosis as well as the NASH activity index. Hence, formulations according to embodiments herein incorporate Bifidobacterium bifidum.

Prebiotics is a general term to refer to chemicals that induce the growth and/or activity of commensal microorganisms (e.g., bacteria and fungi) that contribute to the well-being of their host. A prebiotic is a selectively fermented ingredient that allows specific changes, both in the composition and/or activity in the gastrointestinal microflora that confers benefits upon host well-being and health. Prebiotics include trans-galactooligosaccharide and inulin. The most common example is in the gastrointestinal tract, where prebiotics can alter the composition of organisms in the gut microbiome. However, in principle it is a more general term that can refer to other areas of the body as well. For example, certain hand moisturizers have been proposed to act as prebiotics to improve the activity and/or composition of the skin microbiota. In patent application US20110312910 A1, the use of a prebiotic in conjunction with hepatoprotector was hypothesized to effectively treat liver disease. In the embodiments herein, the combination of herbs, probiotics, and prebiotics provide a novel CLD treatment.

In the embodiments, prebiotics include short-chain, long-chain, and full-spectrum prebiotics. “Short-chain” prebiotics, e.g. oligofructose, contain 2-8 links per saccharide molecule and are typically fermented more quickly in the right side of the colon providing nourishment to the bacteria in that area. Longer-chain prebiotics, e.g. inulin, contain 9-64 links per saccharide molecule, and tend to be fermented more slowly, nourishing bacteria predominantly in the left-side colon. Full-spectrum prebiotics provide the full range of molecular link-lengths from 2-64 links per molecule, and nourish bacteria throughout the colon, e.g. Oligofructose-Enriched Inulin (OEI). In embodiments herein, the formulation includes Jerusalem artichoke powder as a prebiotic.

The formulation according to embodiments herein includes a prebiotic, an herbal combination and probiotics, thus preferentially resulting in a symbiotic formulation. A symbiotic formulation includes one (or more) probiotic with a one (or more) prebiotic wherein the prebiotic and probiotic are complementary and synergistic, thus presenting a multiplicative factor on their individual actions.

It has been shown that the use of a symbiotic formulation may promote an increase in the number of bifidobacteria, glycemic control, reduction of blood cholesterol, balancing the intestinal flora which aids in reducing constipation and/or diarrhea, improves intestinal permeability and stimulation of the immune system. Clinical indications for these products have been expanded, in order to maximize the individual's physiological functions. So, with the high interest in the clinical and nutritional control of disease, many studies have been conducted demonstrating the effectiveness of using symbiotic formulations in improving and/or preventing various and/or symptoms of gastrointestinal diseases.

Additionally, the supplement could be additive free, gluten free (with no corn, no soy, no yeast, no dairy, no ingredients of animal origin), and kosher.

In addition, pharmaceutical ingredients (also referred to as active pharmaceutical ingredient or API) can be added to compositions of the embodiments herein. API refers to a substance or substance combination used in manufacturing a drug product. API also refers to the active or central ingredient in the product which causes the direct effect on the disease diagnosis, prevention, treatment or cure. While API is the active component of a drug, excipients are the inactive or inert substances present in a drug.

APIs can be extracts and purified chemicals, and sometimes referred to as allopathic drugs. APIs can be chemically synthesized.

The compositions of the embodiments herein comprising synthesized, purified or extracted APIs with a combination of a prebiotic, probiotic and a medicinal herb can significantly reduce adverse side effects observed using just the APIs as the medicinal herbs do not produce significant side effects, and can further shield against the side effects observed using just the APIs. This is because the active ingredients are combined with other compounds in the herb and administered in different dosages, and the prebiotic, probiotic and/or a medicinal herb have the effect of shielding against the adverse side effects observed using just the APIs by interfering in the pathways that cause the side effects due to the APIs alone.

The American Cancer Society states on its website: “There is some evidence from randomized clinical trials that some Chinese herbs may contribute to longer survival rates, reduction of side effects, and lower risk of recurrence for some types of cancer, especially when combined with conventional treatment.” However, more research is needed to validate that the combination of herbal medication and APIs can reduce the side effects of the APIs. See also, “COUNTERING THE SIDE EFFECTS OF MODERN MEDICAL THERAPIES WITH CHINESE HERBS,” by Subhuti Dharmananda, Ph.D., Director, Institute for Traditional Medicine, Portland, Oreg., available on-line at http://www.itmonline.org/arts/sidefx.htm.

Milk thistle (Silybum marianum) is an herbal remedy for a variety of ailments, particularly liver, kidney, and gall bladder problems. Several scientific studies suggest that substances in milk thistle (especially a flavonoid called silymarin) protect the liver from toxins, including certain drugs such as acetaminophen (Tylenol), which can cause liver damage in high doses. Silymarin has antioxidant and anti-inflammatory properties, and it may help the liver repair itself by growing new cells. The active ingredient—the one that protects the liver—in milk thistle is known as silymarin, a chemical extracted from the seeds. Silymarin is actually a group of flavonoids (silibinin, silidianin, and silicristin), which are thought to help repair liver cells damaged by alcohol and other toxic substances. Silymarin also keeps new liver cells from being destroyed by these same toxins. It reduces inflammation (which is why it is often suggested for people with liver inflammation or hepatitis) and is a strong antioxidant. Embodiments herein relate to a composition comprising APIs and milk thistle.

Most milk thistle products are standardized preparations made from the seeds of plant. Most preparations are standardized to contain 70-80% of silymarin.

APIs in the compositions of the embodiments herein include any FDA approved APIs, e.g., Statins—pharmaceutical preparation used for lowering cholesterol; Tetracycline (antibiotic)—pharmaceutical preparation clinically used to inhibit pathogenic bacteria, a synthesized or extracted compound; Nifedipine—pharmaceutical used to reduce hypertension, a chemically synthesized compound; Atenolol—pharmaceutical preparation clinically used to reduce hypertension, a synthesized compound; Fungal diastase—pharmaceutical preparation to reduce indigestion, an extracted and purified enzyme; Papain—pharmaceutical preparation used to reduce indigestion, extracted and purified enzyme; and Simethicone (silica compound)—pharmaceutical preparation used to reduce gassiness, chemically synthesized compound.

Some embodiments of the composition disclosed herein include APIs for liver diseases such as nonalcoholic fatty liver disease, and the APIs could be pioglitazone (class: thiazolidinediones); orlistat (class: peripherally acting antiobesity agents); rosiglitazone (class: thiazolidinediones); ursodiol (class: gallstone solubilizing agents); betaine (class: nutraceutical products).

Embodiments also relate to a health supplement, wherein a supplement refers to a composition containing one or more herbs in conjunction with one or more probiotics as well as a prebiotic.

### Supplement Preparation:

One of the methods the supplement can be prepared can be by combining either a decoction or infusion of the herb(s), and adding it to the probiotics by mixing. A decoction is a concentrated solution of the herb made by boiling the root or bark of the herb. An infusion is, similarly, a concentrated solution of the herb made by boiling delicate parts of the herb, such as flowers or leaves. The amount of each herb can be determined by the dosages listed in the above table. The supplement can be supplied as a capsule or a liquid.

Several methods of standardization may be determining the amount of herbs used. One is the ratio of raw materials to solvent. However different specimens of even the same plant species may vary in chemical content. For this reason, thin layer chromatography is sometimes used by growers to assess the content of their products before use. Another method is standardization on a signal chemical.

Below are examples of how toprepare a decoction or infusion:

Preparation of Caper bush:

Coarsely powdered Caper bush plant bark is extracted with ethanol (80% v/v) for five hours, and then filtered. After filtration, the extract is evaporated in a vacuum below 50° C.

### Preparation of Chicory:

Add one cup of water to two teaspoons of Chicory rootstock or dried herb. Boil for ten minutes and strain solid.

### Preparation of Black Nightshade:

Black nightshade extract is prepared from the juice of its leaves. The juice is then warmed until it loses its green color and becomes a reddish brown. It is then cooled and strained and ready for oral administration.

### Preparation of Ginseng

1 L of water is mixed with 10 g to 20 g of ginseng. Put all the ingredients into the cooking pot and cook for an hour or longer, until some of the water evaporates.

### Preparation of Lactobacillus Probiotic

The probiotic can be prepared by the following technique:

A bacterial culture of, for example, Lactobacillus can be added to antibiotic-free milk. Heat one gallon of milk slowly to about 185° F.-195° F. Ensure that the milk does not boil. Maintain this temperature for one hour. Then, allow the milk to cool to the optimal growth temperature of the probiotic. The optimal growth temperature for Lactobacillus is 99° F. Add one cup of the Lactobacillus culture to one cup of the cooled milk. Stir this mixture into the remaining gallon of milk. Allow the mixture to remain at the optimal growth temperature (99° F.) for 24 hours, after which the probiotic should be refrigerated.

### Preparation of Lactobacillus and Caper Bush Probiotic Herbal Mixture

Decoctions or infusions of the various herbs can be added to the chosen probiotic by mixing. For example, the appropriate dosage of herbal extract listed in Table 1, (10-80 g/kg body weight for Caper bush) can be added to about 20-90 g (107 organisms per grain) of the Lactobacillus probiotic. The herbal extract and the probiotic can be combined into a liquid mixture.

Table 2 shows three formulations of the embodiments of the invention for low, moderate and strong dosage formulations.

TABLE 2
Herbal ingredients and formulations

| Herbal preparation | Formulation #1 | | Formulation #2 | | Formulation #3 | |
|---|---|---|---|---|---|---|
| Caper bush or C. spinosa | 30 | g/kg body weight | 45 | g/kg body weight | 60 | g/kg body weight |
| Chicory | 3 | grams per day | 4 | grams per day | 5 | grams per day |
| Black nightshade or S. nigrum | 0.5 | g/kg body weight | 1.0 | g/kg body weight | 1.5 | g/kg body weight |
| Tamarisk | 3 | grams per day | 7 | grams per day | 10 | grams per day |
| Schisandra Chinensis | 1.0 | g/kg body weight | 1.5 | g/kg body weight | 2.0 | g/kg body weight |
| Bupleurum Chinense | 200 | mg/kg body weight | 400 | mg/kg body weight | 500 | mg/kg body weight |
| Ginger | 1 | gram per day | 2 | grams per day | 4 | grams per day |
| Ginseng | 1 | gram per day | 1.5 | grams per day | 2.0 | grams per day |
| Licorice | 1 | gram per day | 2.0 | grams per day | 3.0 | grams per day |
| Turmeric | 50 | mg/kg of body weight | 75 | mg/kg of body weight | 100 | mg/kg of body weight |
| Silymarin | 120 | mg per day | 260 | mg per day | 300 | mg per day |
| Glutathione | 75 | mg per day | 100 | mg per day | 150 | mg per day |
| Neem | 400 | mg/kg body weight | 500 | mg/kg body weight | 600 | mg/kg body weight |
| Astragalus root | 150 | mg/kg of body weight | 175 | mg/kg of body weight | 200 | mg/kg of body weight |
| Choline | 1.0 | gram per day | 2.0 | grams per day | 3.5 | grams per day |
| Crataegus Pinnatifida | 50 | mg/kg of body weight | 75 | mg/kg of body weight | 100 | mg/kg of body weight |
| Probiotic formulation: | | | | | | |
| Lactobacillus acirophilus | 5 | BU | 10 | BU | 15 | BU |
| Lactobacillus rhamnosus | 5 | BU | 10 | BU | 15 | BU |
| Bifidobacterium Bifidum | 5 | BU | 10 | BU | 15 | BU |
| Prebiotic formulation: | | | | | | |
| Raw Jerusalem artichoke powder | 100 | mg | 200 | mg | 300 | mg |

BU means billion units of the microorganism

In yet other embodiments, the amount of probiotics such as Lactobacillus acidophilus, Lactobacillus rhatranosus, and Bifidobacterium Bifidum, in the formulation could range from 0.1 BU to 100 BU, 1 BU to 75 BU, 2 BU, to 50 BU, 4 BU, to 25 BU, and 7.5 BU to 20 BU.

In yet other embodiments, the amount of prebiotic such as raw Jerusalem artichoke power could range from 1 mg to 1000 mg, 10 mg to 800 mg, 25 mg to 600 mg, 50 mg to 500 mg, and 75 mg to 400 mg.

### Supplement Administration:

There are many forms in which the supplement can be administered, the most common of which is in the form of a liquid that is drunk by the patient. Additionally, the supplement can be developed as a pill to be orally administered.

### Supplement Packaging:

All bottles that carry the supplement pills will have anti-counterfeiting technology with digital laser trademarks. The packaging will have covert digital technologies, which cannot be detected by the naked eye and can only be detected electronically. This will also include technology for product tracking and tracing, which can include covert bar codes and serialization.

### EXAMPLES

### Example 1

300 capsules of the herbal formulation having the composition shown below were prepared and tested using ob/ob mice. Each mouse was fed three capsules per day from Formulation #2, shown above, as the middle range dose. Three capsules in total contain the following ingredients by weight in milligram (MG) or billion units (BU):
CHICORY ROOT 30 MG (Manufacturer: Natricargo)
SCHISANDRA BERRY 750 MG (Manufacturer: Natricargo)
BUPLEURUM ROOT 150 MG (Manufacturer: Mountain Rose Herbs)
GINGER ROOT 10 MG (Manufacturer: PCCA)
GINSENG ROOT POWDER (PANAX GINSENG) 10 MG (Manufacturer: PCCA)
LICORICE ROOT POWDER (GLYCYRRHIZA GLABRA) 10 MG (Manufacturer: PCCA)
TURMERIC POWDER (CURCUMA LONGA) 37.5 MG (Manufacturer: PCCA)
MILK THISTLE (SILYBUM MARIANUM) 12 MG (Manufacturer: PCCA)
GLUTATHIONE (L) REDUCED USP 7.5 MG (Manufacturer: PCCA)
ASTRAGALUS ROOT POWDER 1.12.5 MG (Manufacturer: Natricargo)
CHOLINE BITARTRATE USP 10 MG (Manufacturer: PCCA)
JERUSALEM ARTICHOKE POWDER 190 MG (Manufacturer: Natricargo)
LACTOBACILLUS ACIDOPHILUS 10 BU (Manufacturer: UAS Labs)
LACTOBACILLUS RHAMNOSUS 10 BU (Manufacturer: UAS Labs)
BIFIDOBACTERIUM BIFIDUM 10 BU (Manufacturer: UAS Labs)
Each empty capsule weighs 0.097 gm and the contents of one filled capsule weigh 0.48 gm.

### Example 2

### Test Protocol for Testing the Herbal Formulations

PROTOCOL TITLE: Effects of an herbal formulation on NAFLD/NASH development in Black 6 ob/ob mice.

Hypothesis: Black 6 ob/ob mice lack leptin and develop obesity with numerous systemic effects. Their livers are larger, heavier and fattier than normal mice. An herbal formulation may work to ameliorate this lipogenesis, the oxidative stress and resulting fibrogenesis.

Protocol Outline:

1. A control group of 3 female mice that are 12 weeks old were compared to an experimental group of 3 male mice that are also 12 weeks old. The control group was fed a natural ingredient diet 2018S. The groups were separated by gender to demonstrate a more pronounced effect as male ob/ob mice tend to have a greater degree of steatosis compared to females

2. The experimental group of 3 male mice received the herbal formulation of Example 1 three times a day. The herbal formulation was developed in capsules which may be opened and the contents mixed in with the natural ingredient diet. Feedings occurred at baseline, 3 hours later and finally, 6 hours later.

3. Baseline laboratory testing done for ALT, AST, alkaline phosphatase, GGT, cholesterol, triglycerides, glucose.

4. Animals sacrificed at day 30 after bleeding done for repetition of all baseline laboratory testing for comparison.

5. Livers weighed for each animal with histology slides and interpretation done. Oil-red-O stain, H&E stain, Trichrome stain done on all slides. Photomicrographs done, digitized and available.

6. All materials of lab records, laboratory values, slides available.

Histopathology Report—Effects of an herbal formulation on NAFLD/NASH development Black ob/ob mice

### 1.0 Introduction

1.1 Study Objectives
To determine the effects of herbal formulations on non-alcoholic fatty liver disease (NAFLD) and non-alcoholic steatohepatitis (NASH) development in black ob/ob mice.

1.2 Pathology Objectives
To histologically compare the degree of steatosis, inflammation, hepatocellular degeneration, and fibrosis in the livers f treated and untreated black ob/ob mice.

### 2.0 Materials and Methods

2.1 Trimming and Histology
Sample Type: Sagitally bisected murine livers. The left side is submitted fresh in Dulbecco's phosphate-buffered saline. The right side is submitted in fixed 10% neutral buffered formalin.
Processing Request: A cross-section of fresh tissue is to be used for oil red o staining on frozen sections. A cross section of the formalin fixed specimen is to be used for routine paraffin embedding, microscope slide creation, and slide staining with hematoxylin-eosin (H&E) and Masson's trichrome.
Histology Summary: Fresh frozen sections of murine livers were stained with oil red o. Frozen sections of formalin fixed mi stained with a modified oil red o stain. Both sections were considered during histologic assessment. A cross-section for the formalin-fixed right liver lobes was routinely processed to provide H&E and Masson's trichrome stained slides.

Summary:

| | |
|---|---|
| Experiment Name | EB-2016-0001 |
| Total number of | 12 (1 formalin fixed right liver and 1 fresh |
| samples | left liver per animal) |
| Stains | H&E, Masson's trichrome, oil red o |
| Number of | 5 (1 H&E, 1 Masson's trichrome, 1 fresh frozen |
| slides per | oil red o, 1 formalin fixed frozen oil red o, |
| sample | 1 formalin fixed frozen oil red o negative control) |
| Total number | 33 (5 slides/animal, 1 Masson's trichrome positive |
| of slides | control, 1 oil red o positive control, 1 oil red o |
| | negative control) |

2.2 Histopathology Scoring¹ From Brunt E M and Tiniakos D G. Histopathology of nonalcoholic fatty liver disease. 2010. World J Gastroenterol. 16(42): 5286-5296.

The following criteria are scored and used to compare treatment groups:
Fatty change (steatosis)
   0: <5%
   1: 5-33%
   2: 34-66%
   3: >66%
Lobular inflammatory cell infiltrate
   0: absent
   1: <2 foci
   2: 2-4 foci
   3: >4 foci
Portal inflammatory cell infiltrate
   0: absent
   1: mild
   2: moderate
   3: severe
Hydropic (ballooning) degeneration
   0: none
   1: few cells
   2: many cells
Biliary hyperplasia
   0: absent
   1: affects <25% portal triads
   2: affects 26-50% of portal triads
   3: affects 51-75% of portal triads
   4: affects >75% of portal triads
Fibrosis
   0: none
   1a: mild, zone 3 perisinusoidal
   1b: moderate, zone 3 perisinusoidal
   1c: portal/periportal fibrosis only
   2: zone 3 perisinusoidal and portal/periportal fibrosis
   3: bridging fibrosis
   4: fibrosis with nodular/non-nodular regeneration

### 3.0 Histopathology

TABLE 1
Histopathology data organized by treatment group

| | | | | | Lobular | Portal | Hydropic | | |
|---|---|---|---|---|---|---|---|---|---|
| | | | % | % | Inflammatory | Inflammatory | (Ballooning) | Biliary | |
| Sample | Treatment | Steatosis | Macrovesicular | Microvesicular | Infiltrate | Infiltrate | Degeneration | Hyperplasia | Fibrosis |
| ID | Group | (0-3) | Steatosis | Steatosis | (0-3) | (0-3) | (0-2) | (0-4) | (0-4) |
| 1C | Control | 3 | 20% | 80% | 2 | 2 | 1 | 4 | 1c^{#} |
| 6C | Control | 3 | 40% | 60% | 0 | 0 | 0 | 0 | 0 |
| 10C | Control | 3 | 20% | 80% | 0 | 1 | 1 | 3 | 1c^{#} |
| Mean | Control | 3 | 27% | 73% | 0.67 | 1 | 0.67 | 2.33 | 0.67 |
| 2 | Supplement | 3 | 40% | 60% | 0 | 0 | 0 | 0 | 1c^{$} |
| 3 | Supplement | 3 | 50% | 50% | 0 | 0 | 0 | 0 | 0 |
| 9 | Supplement | 2* | 60% | 40% | 0 | 0 | 0 | 0 | 0 |
| Mean | Supplement | 2.67 | 50% | 50% | 0 | 0 | 0 | 0 | 0.33 |

^{#}the majority of portal triads are affected
^{$}only one portal triad is affected
*steatosis more prominent centrilobularly (zone 3)

3.2 Summary of Histologic Findings

### Control Group

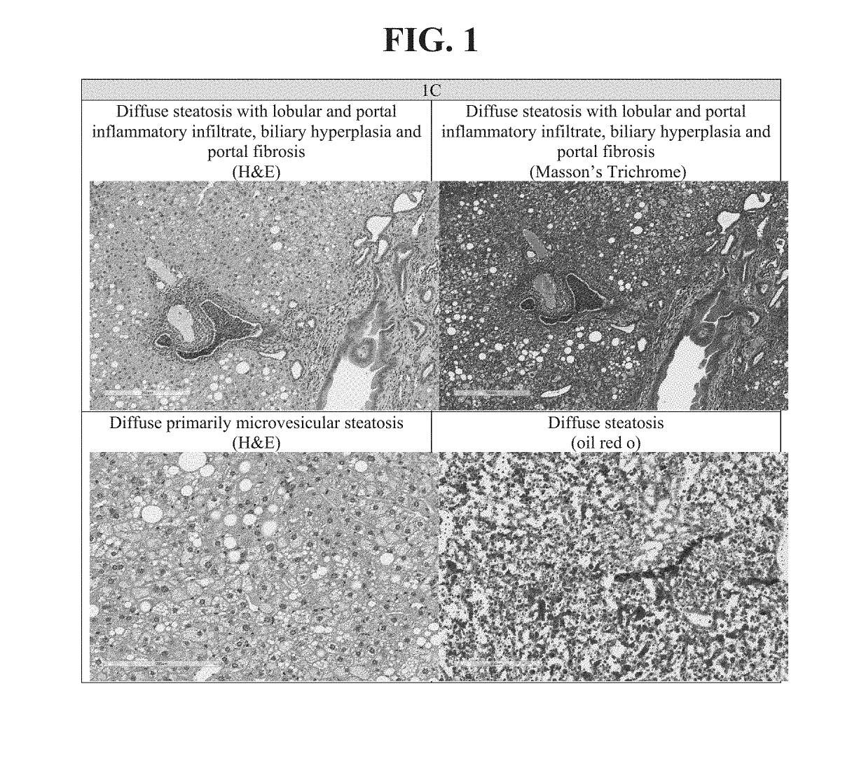

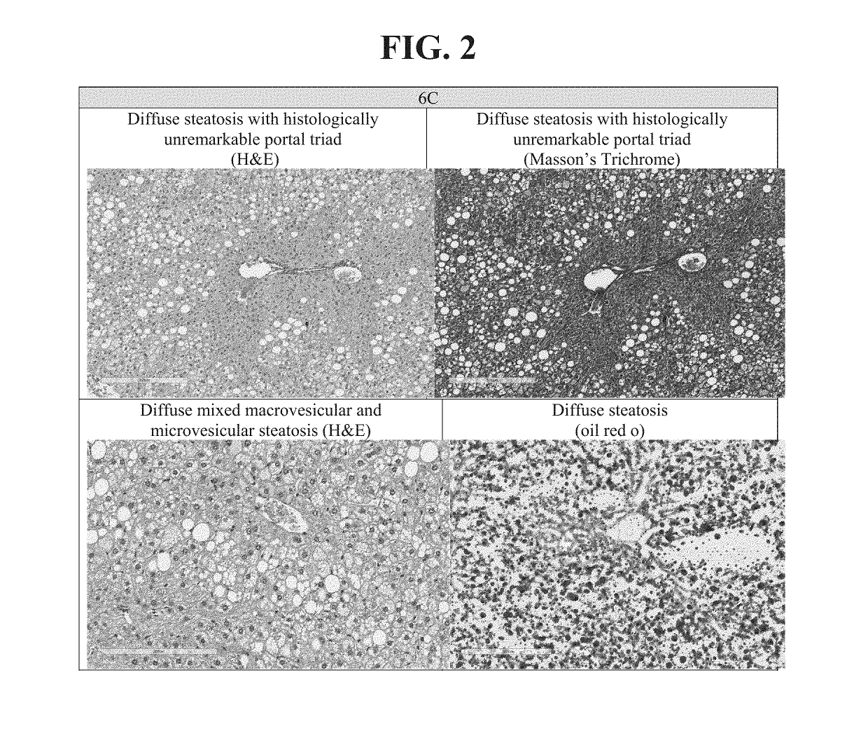

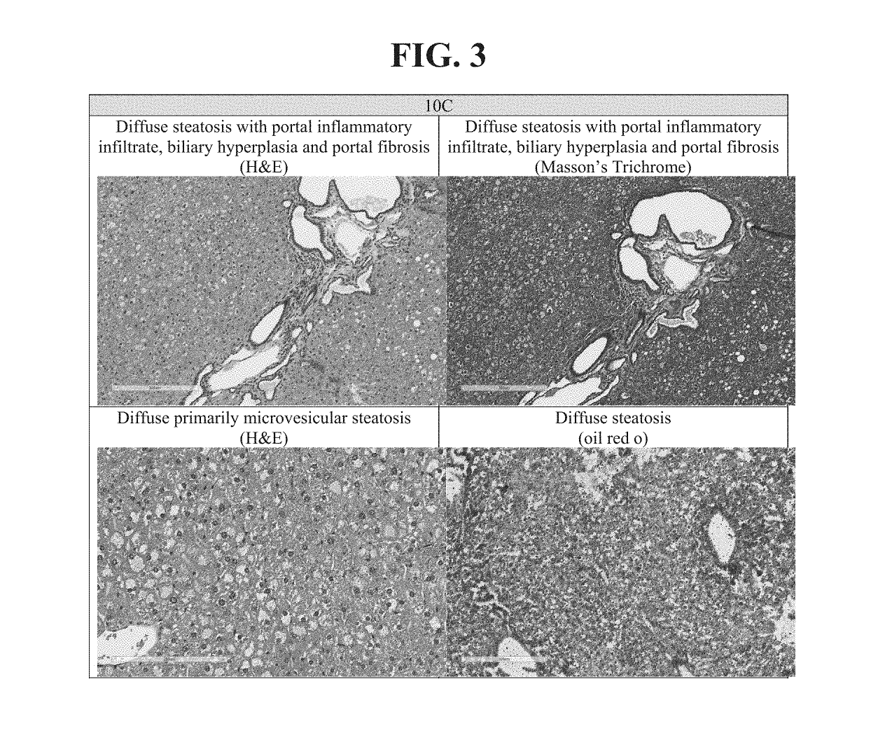

Hepatocellular steatosis is diffuse within the livers of the control animals. Hepatocyte cytoplasm is either distended by a single, large, well-defined, rounded, optically vacant vacuole that displaces the nucleus to the periphery (macrovesicular steatosis) or is partially or completely filled with numerous small, round, punctate, optically vacant vacuoles that do not displace the cytoplasm (microvesicular steatosis). The proportion of macrovesicular and microvesicular steatosis varies within control animal livers but microvesicular steatosis tends to predominate. Liver 1C (FIG. 1) contains multifocal lobular areas of hepatocellular necrosis and loss, degeneration, and neutrophilic; inflammatory infiltrate with mild portal neutrophilic inflammatory infiltrate and moderate portal lymphocytic inflammatory infiltrate. There is evidence of hepatocellular loss and remodeling evidenced by closer approximation of adjacent portal triads. All portal triads contain hyperplastic biliary ductules, some of which are mildly ectatic. Multifocally, binary ductules contain intraluminal neutrophils. Fibrosis is mild and limited to portal areas. Inflammation, binary hyperplasia, and fibrosis are not observed in Liver 6C (FIG. 2). Liver 10C (FIG. 3) is histologically similar to Liver 1C but inflammatory infiltrates and biliary hyperplasia are lessened.

### Supplement Group

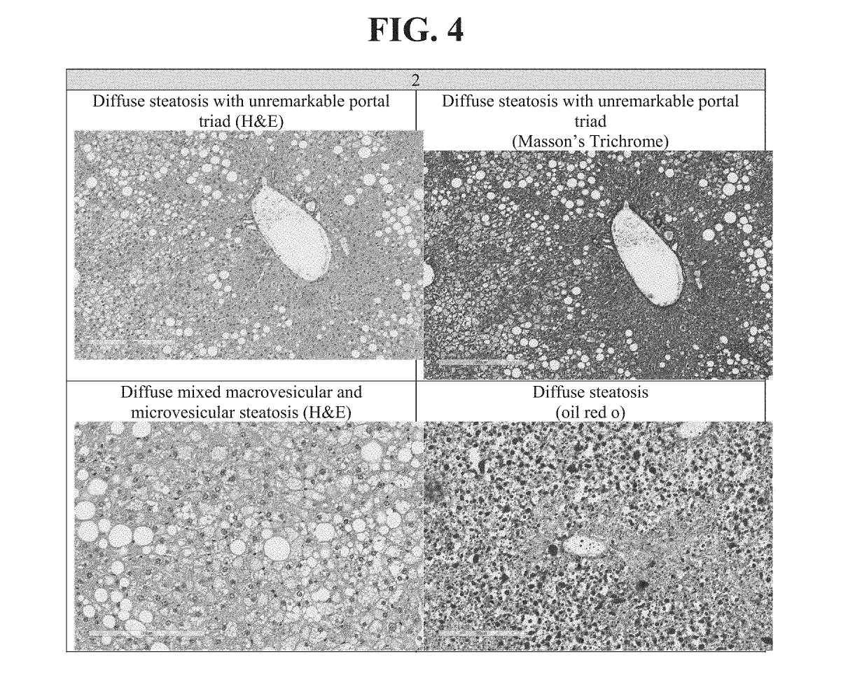

One supplement treated Liver 2 (FIG. 4) contained one portal triad with mild fibrosis.

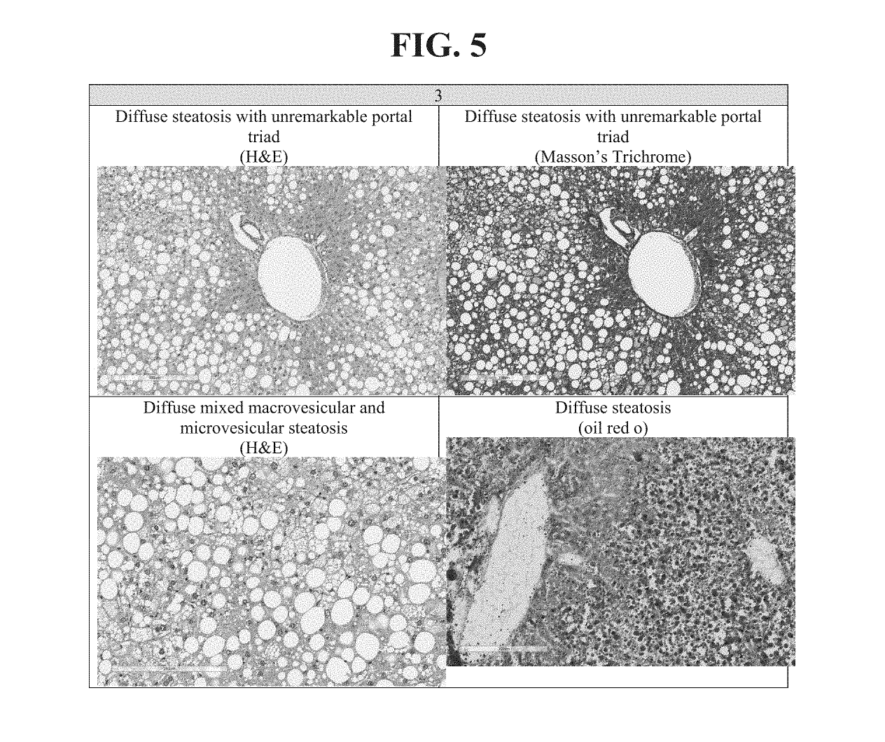

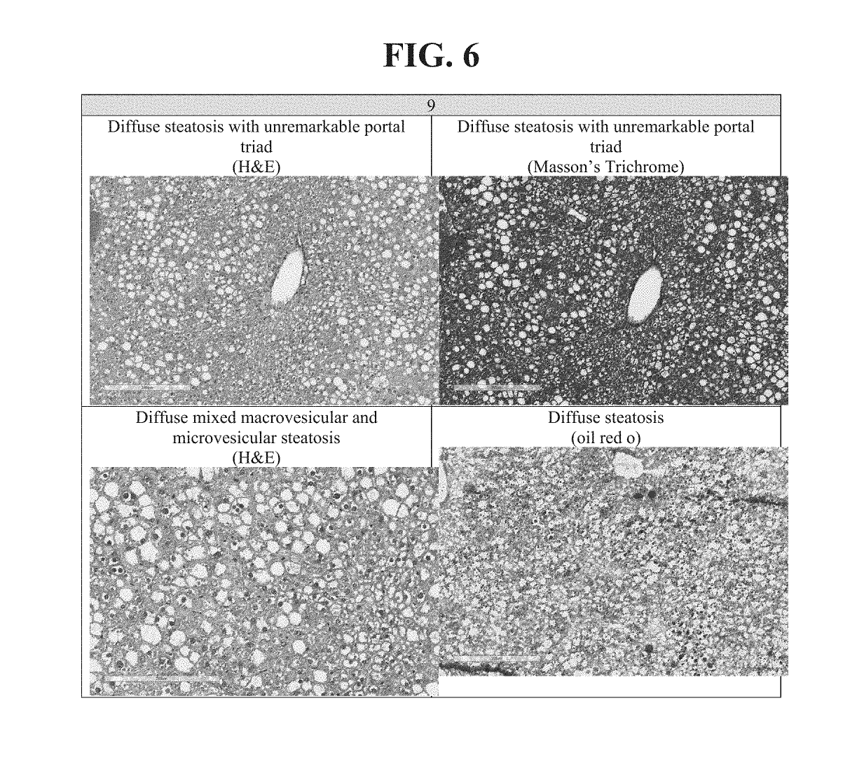

Hepatocellular steatosis is diffuse within the liver of the supplement treated animals; inflammation, degeneration, and biliary hyperplasia are not identified in the supplement treated Livers 3 and 9 (FIGS. 5 and 6). Hepatocyte cytoplasm is either distended by a single, large, well-defined, rounded, optically vacant vacuole that displaces the nucleus to the periphery (macrovesicular steatosis) or is partially or completely filled with numerous small, round, punctate, optically vacant vacuoles that do not displace the cytoplasm (microvesicular steatosis). The proportion of macrovesicular and microvesicular steatosis varies within supplement treated animal livers but they tend to occur in approximately equal proportions. In Liver 9 (FIG. 6), steatosis affects approximately 60% of the liver and is more prominent centrilobularly.

### 4.0 Conclusions

Steatosis is diffuse in both the control and supplement treated mouse livers. Control livers tended to contain relatively more microvesicular steatosis compared to supplement treated mouse livers. Inflammatory cell infiltrate, necrosis, degeneration, biliary hyperplasia, and fibrosis are prominent in two of three control mouse livers but are not prominent in supplement treated mouse livers.

The results are consistent with a benefit in the supplement treated mice which showed less scarring in their livers. There was also a benefit in the treated mice as far as liver enzymes, glucose control and lipids (cholesterol and triglycerides). This has implications beyond the liver to this supplement helping the metabolic syndrome systemically

### Applications and Uses

The formulations of the embodiments herein show an improvement in lab values in glucose, cholesterol and triglycerides in the treated mice versus the controls. This allows wider claims to other manifestations of the “metabolic syndrome.” NASH is the liver mainfestation of the metabolic syndrome. The metabolic syndrome is defined by the National Institutes of Health as the following (http://www.nhlbi.nih.gov/health/health-topics/topics/ms):

Metabolic syndrome is the name for a group of risk factors that raises your risk for heart disease and other health problems, such as diabetes and stroke.

The term “metabolic” refers to the biochemical processes involved in the body's normal functioning. Risk factors are traits, conditions, or habits that increase your chance of developing a disease.

“Heart disease” refers to coronary heart disease (CHD). CHD is a condition in which a waxy substance called plaque builds up inside the coronary (heart) arteries.

Plaque hardens and narrows the arteries, reducing blood flow your heart muscle. This can lead to chest pain, a heart attack, heart damage, or even death.

### Metabolic Risk Factors

The five conditions described below are metabolic risk factors. You can have any one of these risk factors by itself, but they tend to occur together. You must have at least three metabolic risk factors to be diagnosed with metabolic syndrome.
A large waistline. This also is called abdominal obesity or “having an apple shape.” Excess fat in the stomach area is a greater risk factor for heart disease than excess fat in other parts of the body, such as on the hips.
A high triglyceride level (or you're on medicine to treat high triglycerides). Triglycerides are a type of fat found in the blood.
A low HDL cholesterol level (or you're on medicine to treat low HDL cholesterol). HDL sometimes is called “good” cholesterol. This is because it helps remove cholesterol from your arteries. A low HDL cholesterol level raises your risk for heart disease.
High blood pressure (or you're on medicine to treat high blood pressure). Blood pressure is the force of blood pushing against the walls of your arteries as your heart pumps blood. If this pressure rises and stays high over time, it can damage your heart and lead to plaque buildup.
High fasting blood sugar (or you're on medicine to treat high blood sugar). Mildly high blood sugar may be an early sign of diabetes.

Our formulation of a prebiotic, herbal, probiotic, and therefore, a symbiotic, seems to improve blood sugar, cholesterol, triglycerides and therefore, claim can be extended to treatment of metabolic syndrome and its manifestations of diabetes, high lipids and consequent risk for heart disease.

### INCORPORATION BY REFERENCE

All patents, applications and publications referenced in the application are incorporated herein by reference in their entirety.

What is claimed is:

1. A composition comprising a probiotic and a medicinal herb, wherein the probiotic comprises a live o-organism, wherein the composition is effective in preventing or treating inflammation of liver.

2. The composition of claim 1, further comprising a prebiotic, wherein the prebiotic comprises an ingredient that causes specific changes in the composition and/or an activity of the composition in a gastrointestinal microflora so as to confer a therapeutic benefit to the human or the animal.

3. The composition of claim 1, wherein the medicinal herb comprises fennel, fenugreek, or mixtures thereof.

4. The composition of claim 1, wherein the composition further comprises a vitamin, a mineral supplement, a bulking agent, a hydrocolloid, a lipotrophic factor or mixtures thereof.

5. The composition of claim 1, wherein the probiotic comprises Lactobacillus acidophilus, Bifidobacterium bifidus, Lactobacillus rhamnosus or mixtures thereof.

6. The composition of claim 1, wherein the probiotic comprises a non-pathogenic microbial culture.

7. The composition of claim 1, wherein the probiotic comprises a multiple mixed stain culture.

8. The composition of claim 1, wherein the probiotic comprises genera Lactococcus, Lactobacillus, Pediococcus, Streptococcus, Propionibacterium, Brevibacterium, Penicillium, Saccharomyces, or mixtures thereof.

9. The composition of claim 1, further comprising an active pharmaceutical ingredient.

10. The composition of claim 2, further comprising an active pharmaceutical ingredient.

11. The composition of claim 2, wherein the composition is a symbiotic formulation.

12. A composition comprising a prebiotic, a probiotic and a medicinal herb, wherein the composition is effective in preventing or treating a disease or disorder in a human or an animal, wherein the probiotic comprises a live micro-organism, wherein the prebiotic comprises an ingredient that causes specific changes in the composition and/or an activity of the composition in a gastrointestinal microflora so as to confer a therapeutic; benefit to the human or the animal, wherein the disease or the disorder is selected from the group consists of anemia arthritis, constipation, depression, diabetes, dyspepsia, hemorrhoids, hepatitis, hypertension, impotency, obesity, overweight, periodontal disease, tension, memory loss, joint stiffness, joint pain, swelling, liver disease, rectal bleeding, rectal pain, male potency, loss of sex drive, high blood cholesterol, bleeding gums, bad breath, tooth ache, digestive disorder, bowel disorder, and combinations thereof.

13. The composition of claim 11, further comprising an active pharmaceutical ingredient.

14. A method of preventing or treating inflammation of liver in a human or an animal comprising administering to the human or the animal the composition of claim 1, wherein the probiotic is alive when administered.

15. The method of claim 13, wherein the probiotic is selected from the group consisting of non-pathogenic members of the genera Lactococcus, Lactobacillus, Pediococcus, Streptococcus, Propionibacterium, Brevibacterium, Penicillium, and Saccharomyces, and mixtures thereof.

16. The method of claim 13, further comprising, prior to the administering, culturing the probiotic; and drying the, probiotic culture.

17. The method of claim 15, wherein the drying the probiotic culture is by a method selected from the group consisting of spray drying, heat drying, and freeze drying.

18. The method of claim 13, further comprising, prior to the administering, blending ingredients of the composition including the probiotic; and forming the mixture into combined dosage units.

19. The method of claim 13, wherein the administering is performed by administering separate dosage units.

20. The method of claim 13, wherein the composition further comprises a prebiotic, wherein the prebiotic comprises an ingredient that causes specific changes in the composition and/or an activity of the composition in a gastrointestinal microflora so as to confer a therapeutic benefit to the human or the animal.

21. The method of claim 13, wherein the composition further comprises an active pharmaceutical ingredient.
